(19) United States
(12) Patent Application Publication
(10) Pub. No.: US 2019/0374686 A1
(43) Pub. Date: Dec. 12, 2019

(54) METHOD FOR COATING IMPLANT WITH ACTIVE PHARMACEUTICAL INGREDIENTS

(71) Applicant: Ningbo Weston Powder Pharma Coatings Co., Ltd., Ningbo (CN)

(72) Inventors: Chi ZHANG, Shanghai (CN); Jingxu ZHU, London (CA); Yingliang MA, London (CA); Qingliang YANG, Hangzhou (CN); Kaiqi SHI, Ningbo (CN)

(21) Appl. No.: 16/427,604

(22) Filed: May 31, 2019

(30) Foreign Application Priority Data
Jun. 11, 2018 (CN) 201810597273.7

(51) Int. Cl. A61L 31/10; A61K 9/00; A61K 38/14; A61K 47/32; A61K 47/38

(52) U.S. Cl. CPC A61L 31/10; A61K 9/0024; A61K 38/14; A61K 47/32; A61L 2420/02; A61L 2420/06; A61L 2420/08; A61L 2300/406; A61K 47/38

(57) ABSTRACT

Methods for preparing the drug-coated implants include a) providing an implant; b) coating the implant with the first material which includes active pharmaceutical ingredients and the first biocompatible sustained-release polymer materials; c) coating the implant with the second material which includes the second biocompatible sustained-release polymer materials. The methods can be used to prepare a drug-coated implant and a drug coating layer.

### FIELD

The present disclosure provides a method of producing an implant, specifically, coating of an implant with active pharmaceutical ingredients.

### BACKGROUND

With the increasing incidence of trauma, degenerative tumors, and cardiovascular diseases, more and more implants are needed for surgeries, such as internal fixation devices for fractures, the artificial joint prosthesis for the joint replacement, the fusion devices for the spinal operation, and the stent for the treatment of dissecting lesions of large vessels or aneurysm. Orthopedic implants are important in promoting bone healing, restoring the anatomical structure of bone and joint, and improving the stability of bone and joint. The use of scaffolds also reduced the huge risk of previous open operations. But the use of implants also brought many problems such as infection.

At present, there is no ideal method for preventing implant-related infection in the clinic. Systemic antibiotics are commonly used before operations. However, the concentration of antibiotics around the implants is often lower than that in peripheral blood and other tissues because of blood supply disorders in most lesions. To achieve and maintain a higher concentration of antibiotics at the site of infection, the dosage of antibiotics should be increased, which increases the probability of various toxic and side effects of active pharmaceutical ingredients. Local use of antibiotics, such as PMMA beads, is not generally used as a routine preventive measure, and there are limited kinds of loadable antibiotics, in which PMMA cannot degrade and thus results in the small amount of long-term release of antibiotics dosage and other serious shortcomings.

CN104159635A disclosed a drug-coated implant for the cochlea, including a first polymer coating and a second polymer coating on the coated particles, where the active ingredients were present in granular form and covered by a two-layer polymer coating. The first layer of polymer coating was formed by dip coating, air suspension or vapor deposition. The second layer of polymer coating was formed by using polymer solution, and it is required that the active ingredients of the drug and polymer coatings have different solubilities (e.g. they cannot be both hydrophilic or hydrophobic). Coupled with complex coating preparation methods and special requirements for the active ingredients of active pharmaceutical ingredients and polymer properties, the application of this invention was thus restricted.

CN104740692A disclosed a bone internal fixation implant and its preparation methods. The matrix was first prepared by the molding or injection molding technology. Then, a mixed solution which contained the drug carrier and drug was prepared. The mixed solution was sprayed on the matrix by atomizing spraying. Or applying dip coating method to immerse the matrix into the mixed solution to form a drug coating. The premise of this method was to prepare the drug carrier and a mixture of active pharmaceutical ingredients, which required both drug and the drug carrier to be dissolved in water or organic solvent at the same time, which limits the choice of drug and drug carrier. Moreover, the residues of solvents (water or organic solvents, etc.) in the coating layers can lead to infection of the implantation site, thus causing serious toxic and side effects. At the same time, it not only takes much time and power when the solution method is used in drug coatings preparation, but also brings environmental hazards such as discharging of volatile organic compounds (VOC) and waste water.

Therefore, this field needs implants that can solve above problems or improved implants that can realize other purposes and advantages.

### SUMMARY

In order to overcome the shortcomings of the currently used technologies, the present disclosure is proposed.

Firstly, the present disclosure provides a method of producing an implant coated with active pharmaceutical ingredients, comprising:

(a) providing an implant;

(b) applying a first material to the implant, wherein the first material comprises active pharmaceutical ingredients and first biocompatible sustained-release polymer materials;

(c) applying a second material to the implant obtained in step (b), wherein the second material comprises second biocompatible sustained-release polymer materials.

In some embodiments, implants can be made of metals or non-metallic materials.

In some embodiments, the active pharmaceutical ingredients can be water-soluble. In other embodiments, the active pharmaceutical ingredients can be low water-soluble or water-insoluble. In some embodiments, the active pharmaceutical ingredients can be antibiotic, such as Aureomycin. In other embodiments, the active pharmaceutical ingredients can be water-soluble antibiotic.

In some embodiments, the first biocompatible-sustained-release polymer material and the second biocompatible-sustained-release polymer material can be chosen from the following one or any combination thereof: copolymers of ethyl acrylate and methyl methacrylate, vinyl acetate, copolymers of ethyl acrylate and methyl methacrylate, ethyl cellulose, and cellulose acetate. In some embodiments, it can be same for both first biocompatible sustained-release polymer materials and the second biocompatible sustained-release polymer materials. In other embodiments, they can be different for both the first biocompatible sustained-release polymer materials and the second biocompatible sustained-release polymer materials.

In some embodiments, the active pharmaceutical ingredient (drug) is present in a range from about 10 to about 50% w/w, preferably 20-30% w/w, of the total weight of the first material; or/and the said first biocompatible sustained-release polymer materials is present in a range from about 20 to about 60% w/w, preferably 30-50% w/w, of the total weight of the first material.

In some embodiments, the second biocompatible sustained-release polymer materials are present in a range from about 30 to about 90% w/w, preferably 50-80% w/w, of the total weight of the second material.

In some embodiments, the first biocompatible-sustained-release polymer materials and the active pharmaceutical ingredients present in the first material are in the form of micron-sized powder mixture.

In some embodiments, the coating is accomplished by spray coating, preferably by powder spray coating and more preferably by electrostatic powder spray coating.

In some embodiments, a curing step is applied between steps (b) and (c) and/or after steps (c), preferably by heat. For example, materials are heated at a temperature between 50 and 80° C.

In some embodiments, steps (b) and (c) are alternately performed several sets, preferably 2-10 sets, for example 2, 3, 4, 5, 6, 7 8, 9 and 10 sets.

Secondly, the present disclosure provides a drug-coated implant obtained by using the above method.

Thirdly, the present disclosure provides a drug-coated implant that comprises an implant, a first material layer and a second material layer. The first and second material layer are coated on the surface of the implant. The first material contains active pharmaceutical ingredients and first biocompatible sustained-release polymer materials. The second material layer contains the second biocompatible sustained-release polymer materials. The active pharmaceutical ingredients (drugs) are present in a range from about 10 to about 50% w/w, preferably 20-30% w/w, of the total weight of the first material; or/and the said first biocompatible sustained-release polymer materials is present in a range from about 20 to about 60% w/w, preferably 30-50% w/w, of the total weight of the first material. And/or the second biocompatible sustained-release polymer materials are present in a range from about 30 to about 90% w/w, preferably 50-80% w/w, of the total weight of the second material.

In some embodiments, the implant is coated with several sets of alternating first material layer and second material layer, preferably 2-10 sets such as 2, 3, 4, 5, 6, 7, 8, 9 and 10 sets.

Understandably, the implant also comprises the features and their combinations thereof described in the method of the first aspect.

Fourthly, the present disclosure provides an implant coating which comprises a first material layer and a second material layer. The first material layer contains active pharmaceutical ingredients and first biocompatible sustained-release polymer materials. The second material contains the second biocompatible sustained-release polymer materials. The active pharmaceutical ingredients are present in a range from about 10 to about 50% w/w, preferably 20-30% w/w, of the total weight of the first material; or/and the said first biocompatible sustained-release polymer materials is present in a range from about 20 to about 60% w/w, preferably 30-50% w/w, of the total weight of the first material. And/or the second biocompatible sustained-release polymer materials are present in a range from about 30 to about 90% w/w, preferably 50-80% w/w, of the total weight of the second material.

In some embodiments, the implant is coated with several sets of alternating first material layer and second material layer, preferably 2-10 sets such as 2, 3, 4, 5, 6, 7, 8, 9 and 10 sets.

Understandably, the coating of the implant also comprises the features and their combinations thereof described in the method of the first aspect.

Fifthly, the present disclosure provides a method of using the implant, including implanting the implant into an individual in need.

Sixthly, the present disclosure provides the application of the coating described in the fourth aspect in producing an implant.

A further understanding of the functional and advantageous aspects of the invention can be realized by reference to the following detailed description and drawings.

### BRIEF DESCRIPTION OF THE DRAWINGS

Embodiments disclosed herein will be more fully understood from the following detailed description thereof taken in connection with the accompanying drawings, which form a part of this application, and in which:

FIG. 1 shows a schematic diagram of an implant sample of the present disclosure.

FIG. 2 shows a schematic diagram of the spray coating and curing process for an implant.

FIG. 3A and 3B show the embodiment of the drug-coated implant where FIG. 3A shows an implant that contains a set of the first material layer and the second material layer; and

FIG. 3B shows an implant that contains two sets of the first and second material layers.

FIG. 4 shows a dissolution profile of drug-coated implants where Sample 1 and sample 2 represent the implant which contains the first material layer and the second material layer respectively; Samples 3-9 represent the implants which have several sets of first material and second material layers.

### DETAILED DESCRIPTION

Without limitation, the majority of the systems described herein are directed to drug coated implant with active pharmaceutical ingredients. As required, embodiments of the present invention are disclosed herein. However, the disclosed embodiments are merely exemplary, and it should be understood that the invention may be embodied in many various and alternative forms.

The accompanying figures, which are not necessarily drawn to scale, and which are incorporated into and form a part of the instant specification, illustrate several aspects and embodiments of the present disclosure and, together with the description therein, serve to explain the principles of the simulation apparatus. The drawings are provided only for the purpose of illustrating select embodiments of the apparatus and as an aid to understanding and are not to be construed as a definition of the limits of the present disclosure. For purposes of teaching and not limitation, the illustrated embodiments are directed to surgical simulation apparatus and method of using the same.

As used herein, the term “about”, when used in conjunction with ranges of dimensions, temperatures or other physical properties or characteristics is meant to cover slight variations that may exist in the upper and lower limits of the ranges of dimensions so as to not exclude embodiments where on average most of the dimensions are satisfied but where statistically dimensions may exist outside this region. For example, in embodiments of the present invention dimensions of components of an anastomosis device are given but it will be understood that these are not meant to be limiting.

Firstly, the present disclosure provides a method of producing a drug coated implant with active pharmaceutical ingredients, comprising:(a) providing an implant; (b) applying a first material onto the implant, wherein the first material comprises active pharmaceutical ingredients and first biocompatible sustained-release polymer materials; and (c) applying a second material onto the implant, wherein the second material comprises second biocompatible sustained-release polymer materials.

The term “implant” as used herein includes all the metals and non-metallic materials that can be implanted or partially implanted in human or animal body, including but not limited to, medical steel plates, screws, intramedullary nails, artificial joints metals, non-metallic prosthesis, steel cables, Kirschner wires, Steinmann pins, vascular metal stents, intestinal stents, vascular clamps, allogeneic bones, xenogeneic bones, similar veterinary materials, etc.

In some embodiments, the implant can be made of metal material, such as a titanium alloy commonly used in orthopedic implants, cobalt chromium alloys, etc. In other embodiments, the implant can be made of non-metallic material.

It should be understood that the material, shape, size, etc. of the implant are not particularly limited in the present application. Technicians skilled in the art can select an implant which has suitable properties according to a specific application.

The term “drug” as used herein refers to the active pharmaceutical ingredients.

In some embodiments, the active pharmaceutical ingredients are water-soluble. Good water-soluble active pharmaceutical ingredients (such as antibiotics) are more conducive to drug release. However, it should be understood that the active pharmaceutical ingredients may also be low water-soluble active pharmaceutical ingredients or a water-insoluble active pharmaceutical ingredients. And in some embodiments, the active pharmaceutical ingredients are antibiotics, such as vancomycin, cefuroxime, and tobramycin. Useful antibiotics include, but not limited to, amoxicillin, clindamycin, polymyxin, erythromycin, streptomycin, cefazolin, ticarcillin, lincomycin, methicillin, amika Star, etc. These antibiotics are relatively stable in vitro and are commonly used antibiotics for studies of sustained-release topical active pharmaceutical ingredients. And in some embodiments, the active pharmaceutical ingredients are water-soluble antibiotics.

It should be understood that the active pharmaceutical ingredients may be any drugs other than anti-infective active pharmaceutical ingredients. For example, bone growth implants (e.g., steel plates or allogeneic bones) can be used to promote fracture healing. Rapamycin or paclitaxel can be used in vascular stents to inhibit the proliferation of vessel smooth muscle cells, hence preventing the reocclusion of vascular stents. Useful active pharmaceutical ingredients include, but are not limited to, anti-inflammatory active pharmaceutical ingredients, anticoagulants, anti-tumor active pharmaceutical ingredients, etc.

The term “biocompatible sustained-release polymer material” used herein includes biocompatible polymer materials used in the preparation of controlled release and sustained release pharmaceuticals in the pharmaceutical field. These materials are well known to technicians skilled in the art. In some embodiments, the first biocompatible sustained release polymeric materials and the second biocompatible sustained-release polymer materials are selected from any one or any combination of the following: copolymer of ethyl acrylate and methyl methacrylate, vinyl acetate copolymer, copolymer of ethyl acrylate and methyl methacrylate, ethyl cellulose, cellulose acetate. Useful polymer materials also include mixtures of one or more of the following: Ethylene acid polymer, acrylic polymer, fluorine polymer, polyurethane, polyolefin, glycolide, lactide, glycolide/lactide copolymer, polyglycolide, polylactide, methyl lactate, Ethyl lactate, isopropyl lactate, propyl lactate, butyl lactate, octyl lactate, lactitol, lactitol mixture, aluminum lactate, iron lactate, magnesium lactate, manganese lactate, zinc lactate, polyamino acid, polyphosphate, Biological apatite, heparinized polymer, heparin and polylactic acid (PLA). In some embodiments, they can be same for both the first biocompatible sustained-release polymer materials and the second biocompatible sustained-release polymer materials. In other embodiments, it can be different for both the first biocompatible sustained-release polymer materials and the second biocompatible sustained-release polymer materials.

In some embodiments, the active pharmaceutical ingredient is present in a range from about 10 to about 50% w/w, preferably 20-30% w/w, e.g. 10%, 15%, 20%, 25%, 30%, 35%, 40%, 45% or 50%, of the total weight of the first material. In some embodiments, it is present in a range from about 20 to about 60% w/w, preferably 30-50% w/w, e.g. 20%, 25%, 30%, 35%, 40%, 45%, 50%, 55% or 60%, of the total weight of the first material.

In some embodiments, the second biocompatible sustained-release polymer materials are present in a range from about 30 to about 90% w/w, preferably 50-80% w/w, e.g. 30%, 35%, 40%, 45%, 50%, 55%, 60%, 65%,70%, 75%, 80%, 85% or 90%, of the total weight of the second material.

In some embodiments, the first biocompatible sustained-release polymer materials and the active pharmaceutical ingredients present in the first material are in the form of micron-sized powder mixture. The advantages of micron-sized powder mixture, in the present application, are forming a dense and sustained release polymer film which has smooth and stronger appearance at a low temperature (less than 60° C.) through solid powder coating.

In some embodiments, the coating is accomplished by spray coating, preferably by powder spray coating and more preferably by electrostatic powder spray coating. In some embodiments, the first material and/or the second material are applied to the entire surface of the implant. And in other embodiments, only a portion of the surface of the implant is coated with the first material and/or the second material.

In some embodiments, a step of curing is included between step (b) and (c), and/ or after step (c). Curing is preferably carried out by heating, for example, curing the materials at 50-80° C. In some embodiments, to introduce a frictional effect during curing process can further increase the density and the uniformity of the coating layer.

The friction effect described in the present application refers to the introduction of a certain friction between the coated implant and the relatively soft fibrous body in the step of curing, but the coating of the implant cannot be damaged by the friction effect. This relatively gentle friction not only helps to form a dense and good-looking coating but also reduces the curing time.

In some embodiments, before performing steps (b) and/or (c), a step of applying plasticizer can be included, such as spraying triethyl citrate (TEC) to the surface of implant.

In some embodiments, in order to achieve a better sustained release rate and concentration of the active pharmaceutical ingredients, steps (b) and (c) are alternately performed several times, preferably 2-10 sets (times), for example 2, 3, 4, 5, 6, 7, 8, 9 and 10 times. It should be understood that in the method disclosed in the present application, the step numbers (b) and (c) are used for convenience sake only, and they do not intend to show the order in which the steps are performed. In addition, it should also be understood that steps (b) and (c) are not only included in “multiple alternating steps (b) and (c)” or “multiple sets of alternating first material layer and second material layer”, but also included in variations thereof described in the present application. The continuous alternating of the first material layers and the second material layers not only includes the alternating pattern such as: (a), (b), (a), (b), etc., but also includes any other variant combination of alternating patterns, such as: (a), (b), (b), (a), (b), (b) or (a), (a), (b), (a), (a), (b), etc.

Secondly, the present disclosure provides a drug-coated implant obtained by using the above method.

Thirdly, the present disclosure provides a drug-coated implant that comprises an implant, a first material layer and a second material layer. The first and second material layer are coated on the surface of the implant. The first material contains active pharmaceutical ingredients and first biocompatible sustained-release polymer materials. The second material layer contains the second biocompatible sustained-release polymer materials. The active pharmaceutical ingredients (drugs) are present in a range from about 10 to about 50% w/w, preferably 20-30% w/w, of the total weight of the first material; or/and the said first biocompatible sustained-release polymer materials is present in a range from about 20 to about 60% w/w, preferably 30-50% w/w, of the total weight of the first material. And/or the second biocompatible sustained-release polymer materials are present in a range from about 30 to about 90% w/w, preferably 50-80% w/w, of the total weight of the second material.

In some embodiments, the implant comprises several sets of alternating first material layers and second material layers, preferably 2-10 sets, such as 2, 3, 4, 5, 6, 7, 8, 9, and 10 sets.

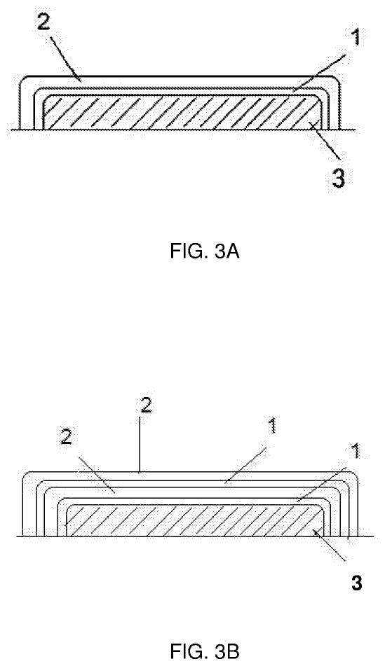

FIGS. 3A and 3B illustrate an embodiment of a drug-coated implant of the present application. The implant 3 illustrated in FIG. 3A includes one set of first material layer 1 and second material layer 2. The implant 3 shown in FIG. 3B comprises two sets of first material layers 1 and second material layers 2. The shaded portions in FIGS. 3A and 3B are implants 3. And arrows 1 and 2 indicate the first material layer and the second material layer, respectively. Understandably, the implant also comprises the features and their combinations thereof described in the method of the first aspect.

Fourthly, the present disclosure provides an implant coating which comprises a first material layer and a second material layer. The first material layer contains active pharmaceutical ingredients and first biocompatible sustained-release polymer materials. The second material contains the second biocompatible sustained-release polymer materials. The active pharmaceutical ingredients are present in a range from about 10 to about 50% w/w, preferably 20-30% w/w, of the total weight of the first material; or/and the said first biocompatible sustained-release polymer materials is present in a range from about 20 to about 60% w/w, preferably 30-50% w/w, of the total weight of the first material. And/or the second biocompatible sustained-release polymer materials are present in a range from about 30 to about 90% w/w, preferably 50-80% w/w, of the total weight of the second material.

In some embodiments, the implant is coated with several sets of alternating first material layer and second material layer, preferably 2-10 sets such as 2, 3, 4, 5, 6, 7, 8, 9 and 10 sets.

Understandably, the coating of the implant also comprises the features and their combinations thereof described in the method of the first aspect.

Fifthly, the present disclosure provides a method of using the implant, including implanting the implant into an individual in need.

Sixthly, the present disclosure provides the application of the coating described in the fourth aspect in producing an implant.

It will be understood that compounds used in the art of pharmaceutical formulations generally serve a variety of functions or purposes. Thus, if a compound named herein is mentioned only once or is used to define more than one term herein, its purpose or function should not be construed as being limited solely to that named purpose(s) or function(s).

The present process will now be illustrating using the following non-limiting examples.

### EXAMPLES

The following examples are provided to further describe the present application without any limitation.

### Materials and Methods

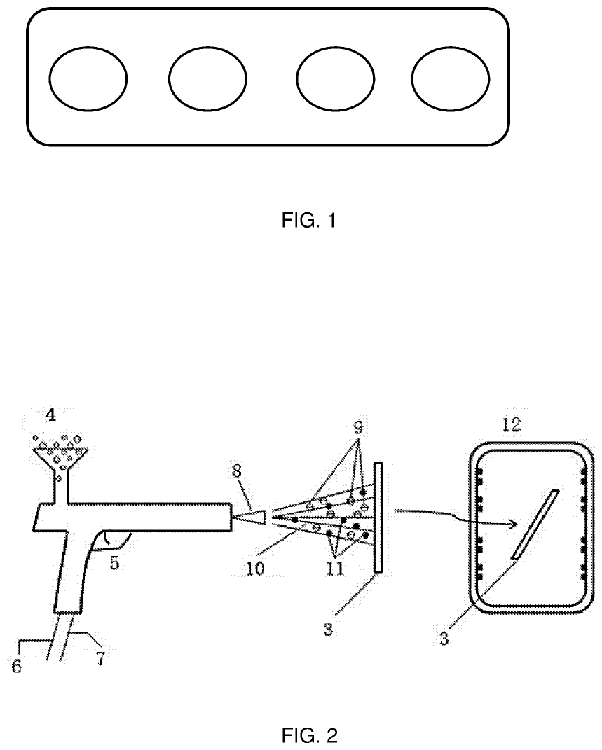

The implant used in the examples, as shown in FIG. 1, is a metal implant hook (20 mm*5 mm).

The formula of the first material is as follow:
a. Copolymer of ethyl acrylate and methyl methacrylate (Eudragit RS), which account for 50% of the total weight of the first material;
b. Vancomycin, which accounts for 30% of the total weight of the first material;
c. The rest are biocompatible materials such as talc and titanium dioxide.

The formula of the second material is as following:
a. Copolymer of ethyl acrylate and methyl methacrylate (Eudragit RS/Eudragit RL), which accounts for 80% of the total weight of the second material;
b. The rest are biocompatible materials such as talc and titanium dioxide.

The electrostatic powder coating method is employed in the examples. FIG. 2 shows the spraying and curing process.

The detailed description of the electrostatic powder coating is as follows with reference to FIGS. 2 and 3A and 3B.

First, the implant 3 is preheated in an oven 12 at 60° C. for 5-10 minutes. After grounding the implant 3 a liquid spray gun 5 is used to spray a suitable amount of coating powder, triethyl citrate (TEC), onto the surface of the implant 3. In this example, an electrostatic spray gun 5 (40-80 kv), is used to spray the powders of the first material onto the surface of the implant 3. Referring to FIG. 2, the spray gun 5 is shown with a powder supply port 4, the voltage line 6, air line 7 and a gun nozzle 8. After heating and curing at 60° C. for some time (30 minutes), a first material layer 1 is formed.

After completing the coating of first material layer 1, the second material layer 2 is coated. First, using a liquid spray gun 5 to spray a suitable amount of triethyl citrate (TEC) onto the surface of the implant 3. And then, using an electrostatic spray gun (40-80 kv) to spray the powders of the second material onto the surface of the implant. After heating and curing at 60° C. for some time (30 minutes), a second material layer 2 is formed afterwards. Repeating the above steps to make the first material layer 1 and the second material layer 2 respectively to meet the corresponding requirements of weight gain.

The use of an electrostatic spray gun 5 can greatly improve the uniformity and controllability of powder coating. The sharp needle electrode, at the front end of the spray gun, ionizes air 11 and negatively charges the particles 9. At the moment, an instantaneous high-voltage electric field 10 is formed between the electrostatic spray gun (40-80 kV) and the grounded implant. Under the effect of electric field force, the negatively charged powders 9 spray towards the substrate (the implant 3) forming a stable deposited layer on the surface of substrate.

There are several advantages of preparing implant coating through electrostatic spray coating. First, it is a dry process when preparing the implant coating through electrostatic spraying. This process avoids using water, organic solvents, etc., whereas the atomized spraying and dip coating method is completely dependent on using water, organic solvents, etc. Therefore, the electrostatic spray coating method avoids discharging waste water and volatile organic compounds which have negative effects on the environment. Besides, this method significantly reduces the manufacturing costs by eliminating the need of using fluidized hot gas to carry water or organic solvents away in the coating process. More importantly, bubbles are easily formed in the coating layers when water or organic solvents evaporate, which affects the uniformity of coating thickness. As a result, the coating layer, being formed in a dry condition without using any water and organic solvent, is more stable and compact. There is absolutely no residues of water or organic solvents, which fundamentally eliminate toxic side-effects such as infections caused by waste water or organic solvents.

Another great advantage of electrostatic spray coating is that the thickness and uniformity of coating layer can be precisely controlled by adjusting some parameters such as the particle size and formula ratio of the first and second material powder composition, the voltage of electrostatic spray gun, etc.

Example of Samples Preparation 1—Single Set of Spraying

The implant was preheated in an oven at 60° C. for 5-10 minutes. Then, a suitable amount of triethyl citrate (TEC) is sprayed onto the surface of the implant, followed by using an electrostatic spray gun (50 kV) to spray the powders of the first material onto the surface of the implant. After heating and curing at 60° C. for some time (30 minutes), a first material layer is formed. The weight gain is 0.8-1.0% after spraying. After that, under the same operation conditions, the triethyl citrate (TEC) and the second material are sprayed onto the surface of the implant. After heating and curing for 30-60 minutes, a second material layer is formed. The gaining weight of the implant is 2.5-3.2%.

Samples 1 and 2 obtained by the above single set of spraying were subjected to drug sustained release test in the following test of samples.

Example of Samples Preparation 2—Multiple Sets of Spraying

The samples 3-9 were obtained by repeating the sample preparation methods which are used in Example 1 under the same operation conditions. This process is similar to the preparation of sample 1 and sample 2, that the implant is first preheated in an oven at 60° C. for 5-10 minutes, and then subjected to an electrostatic spray process. In the electrostatic spray process, a suitable amount of triethyl citrate (TEC) was sprayed onto the surface of the implant, followed by using an electrostatic spray gun (50 kV) to spray the powders of the first material onto the surface of the implant. After heating and curing at 60° C. for some time (30 minutes), a first material layer was formed. After that, under the same operation conditions, the triethyl citrate (TEC) and the second material were sprayed onto the surface of the implant. After heating and curing for 30-60 minutes, a second material layer was formed. The spraying process was repeated until the first and second materials meet the need of gaining weight.

The drug sustained release tests were carried out and the results are shown below. After several sets of spraying, the gaining weight of vancomycin can reach 2.0-2.5%, and the gaining weight of the sustained-release polymer material is 4.0-5.3%. By adjusting the number of spraying times of the first material, different requirements for the content of vancomycin can be achieved. The drug release rate can also be changed by adjusting the number of spraying times of the second material.

Examples of Sample Tests

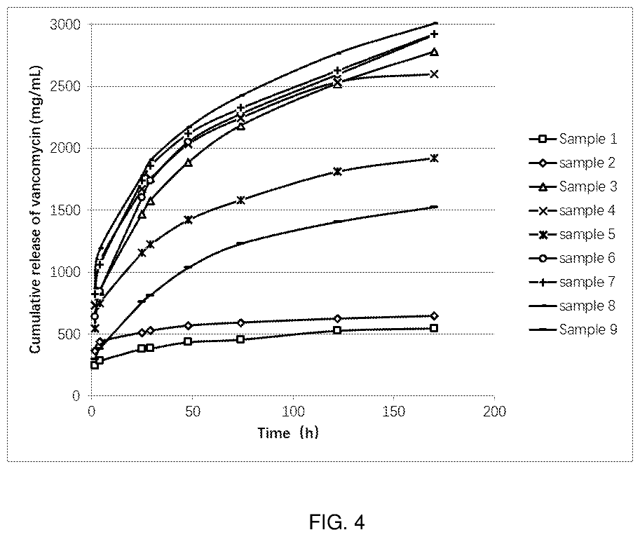

The prepared samples 1-9 were placed in PBS buffer (10 ml) at pH 7.4 and incubated at 37° C. The samples were taken at different time points for HPLC analysis to determine the concentration of vancomycin in the buffer. The experimental results are shown in Table 1 and FIG. 4.

The experimental results show that the effect of sustained release of vancomycin can be achieved by both single set of spraying and multiple sets of spraying. The effect of multiple sets of spraying is more ideal, which not only enables the release of vancomycin for more than one week, but also meets the concentration for antibacterial requirements.

In conclusion, the above embodiments are used only for the purpose of illustrating the technical solutions of the present disclosure but the inventive subject matter of the present disclosure is not limited thereto. Although, the present disclosure has been described in detail which refers to the foregoing embodiments, technicians skilled in the art should understand that the technical solutions or some parts of them still can be modified or equivalently substituted.

These modifications or substitutions do not depart from the scope of the embodiments of the present disclosure.

TABLE 1
The accumulated release time of vancomycin

| Time | Sample | Sample | Sample | Sample | Sample | Sample | Sample | Sample | Sample |
|---|---|---|---|---|---|---|---|---|---|
| 1.5 | 248.4 | 362.4 | 560.6 | 734.6 | 546.4 | 640.8 | 823.8 | 868.6 | 294 |
| 4 | 285.6 | 437 | 844.2 | 1089.2 | 746.8 | 847.4 | 1062.6 | 1189.2 | 382.6 |
| 25 | 382.8 | 513.8 | 1470.6 | 1674.2 | 1158.4 | 1606.2 | 1738.8 | 1781.2 | 759.2 |
| 29 | 384.8 | 527.8 | 1576.2 | 1754.4 | 1222.4 | 1738.6 | 1860 | 1900.8 | 812.4 |
| 48 | 436.2 | 568.6 | 1885.6 | 2034.6 | 1423 | 2053 | 2120.4 | 2168.8 | 1035.8 |
| 74 | 456.6 | 592.6 | 2182.4 | 2245.2 | 1583.8 | 2277.8 | 2325.4 | 2423.4 | 1228 |
| 122 | 527.4 | 624.6 | 2522.4 | 2534 | 1813.6 | 2595.8 | 2629.8 | 2766 | 1405.4 |
| 170 | 546 | 646.4 | 2778 | 2598 | 1923.2 | 2916 | 2924.6 | 3005.2 | 1523.8 |

Therefore what is claimed is:

1. A method of producing a coated medical implant, comprising:
(a) providing an implant;
(b) coating the implant with a first material, wherein the first material comprises active pharmaceutical ingredients and first biocompatible sustained-release polymer materials;
(c) coating the implant obtained in (b) with a second material, wherein the second material comprises second biocompatible sustained-release polymer materials.

2. The method according to claim 1, wherein the said implant is made of metal.

3. The method according to claim 1, wherein said active pharmaceutical ingredients are water-soluble.

4. The method according to claim 1, wherein said active pharmaceutical ingredients are antibiotics.

5. The method according to claim 4, wherein said active pharmaceutical ingredients are vancomycin.

6. The method according to claim 1, wherein said first biocompatible sustained-release polymer materials and the second biocompatible sustained-release polymer materials are any one of the following or any combination thereof: copolymer of ethyl acrylate and methyl methacrylate, vinyl acetate copolymer, ethyl acrylate and methyl methacrylate copolymer, ethyl cellulose, cellulose acetate.

7. The method according to claim 1, wherein said active pharmaceutical ingredients is present in a range from about 10 to about 50% w/w of the total weight of the first material; or/and said first biocompatible sustained-release polymer materials is present in a range from about 20 to about 60% w/w of the total weight of the first material.

8. The method according to claim 7, wherein the active pharmaceutical ingredients is present in 20-30% w/w of the total weight of the first material;

9. The method according to claim 7, wherein the first biocompatible sustained-release polymer materials is present in 30-50% w/w of the total weight of the first material.

10. The method according to claim 1, wherein said second biocompatible sustained-release polymer materials is present in a range from about 30-90% w/w of the total weight of the second material.

11. The method according to claim 10, wherein the second sustained-release polymer materials is present in a range 50-80% w/w of the total weight of the second material.

12. The method according to claim 1, wherein said first biocompatible sustained-release polymer materials and said active pharmaceutical ingredients present in the first material are in a form of micron-sized powder mixture.

13. The method according to claim 1, wherein said coating is accomplished by spray coating.

14. The method according to claim 13, wherein the spray coating is powder spray coating or electrostatic powder spray coating.

15. The method according to claim 1, wherein a curing step is included between steps (b) and (c) and/or after step (c).

16. The method according to claim 15, wherein the curing step is carried out by heating at 50-80° C.

17. The method according to claim 1, wherein steps (b) and (c) are performed alternately several sets.

18. The method according to claim 17, wherein steps (b) and (c) are performed alternately 2-10 sets.

19. An implant produced by the method according to claim 1.

20. A coated implant, comprises an implant, a first material coating layer and a second material coating layer on the surface of the said implant, wherein the said first material coating layer comprises active pharmaceutical ingredients and first biocompatible sustained-release polymer materials and the said second material coating layer comprises the second biocompatible sustained-release polymer materials, wherein:
the said active pharmaceutical ingredients is present in a range from about 10 to about 50% w/w, preferred 20-30% w/w, of the total weight of the first material; or/and the said first biocompatible sustained-release polymer materials is present in a range from about 20 to about 60% w/w, preferably 30-50% w/w, of the total weight of the first material, and/or
the said second biocompatible sustained-release polymer materials is present in a range from about 30 to about 90% w/w, preferably 50-80% w/w, of the total weight of the second material.

21. The implant according to claim 20 is coated with several sets of alternating first material layer and second material layer from at least two (2) sets to about ten (10) sets.

22. An implant coating comprises a first material layer and a second material layer, wherein the first material comprises active pharmaceutical ingredients and first biocompatible sustained-release polymer materials, and the second material comprising second biocompatible sustained-release polymer materials, wherein:
the said active pharmaceutical ingredients is present in a range from about 10 to about 50% w/w, preferred 20-30% w/w, of the total weight of the first material; or/and the said first biocompatible sustained-release polymer materials is present in a range from about 20 to about 60% w/w, preferably 30-50% w/w, of the total weight of the first material, and/or
the said second biocompatible sustained-release polymer materials is present in a range from about 30 to about 90% w/w, preferably 50-80% w/w, of the total weight of the second material.

23. The implant according to claim 20 being coated with several sets of alternating first material layer and second material layer from at least two (2) sets to about ten (10) sets.
